(19) United States
(12) Patent Application Publication
(10) Pub. No.: US 2004/0047873 A1
(43) Pub. Date: Mar. 11, 2004

(54) Materials and methods relating to the increase in protein activity

(76) Inventors: Aymen Al- Shamkhani, Southampton (GB); Martin Glennie, Southampton (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/312,374

(22) PCT Filed: Jun. 25, 2001
(86) PCT No.: PCT/GB01/02810

(30) Foreign Application Priority Data
Jun. 24, 2000 (GB) 0015426.0

(51) Int. Cl. A61K 39/00

(52) U.S. Cl. 424/185.1

(57) ABSTRACT

The invention provides a protein framework which allows active polypeptides e.g. ligands or antigens to be displayed at increased concentration. The inventors show that the lectin binding domains of collectins can be replaced by a polypeptide of interest and that polypeptide can be multimerised by the framework of the collectin and as a result displayed in greater number on a single structure. The inventors show that the activity of polypeptides such as those of the TNF superfamily are significantly enhanced when displayed in this way.

### FIELD OF THE INVENTION

The present invention concerns materials and methods relating to the increase in protein activity. Particularly, but not exclusively, the invention provides a protein framework which is capable of displaying a plurality of associated polypeptides/proteins as a single complex. Further, the invention provides constructs for producing the complexes and the use of these complexes in methods of medical treatment.

### BACKGROUND OF THE INVENTION

The use of proteins or fragments of proteins in methods of medical treatment is increasing in line with the increased knowledge of protein structure and function. For example, many enzymes, antibodies, receptor/ligands, antigens etc are being discovered and their respective functions may have important roles in the treatment or prevention of diseases. Further, insight into the mechanisms of protein-protein interactions provides important information as to how protein drugs may be used to enhance the body's natural defence system against disease. However, in order to maximise the effectiveness of proteins as drugs, it is important that they are administered in a form that can interact efficiently and with the greatest effect.

CD40 is an example of a protein that has potential as a medicament. CD40 is a member of the tumour necrosis factor receptor (TNFR) superfamily expressed on a range of cells, including B cells, monocytes, dendritic cells, follicular dendritic cells, thymic epithelial cells, endothelial cells and epithelial cells [1-3]. CD40 interacts with CD154, a membrane glycoprotein belonging to the TNF superfamily, which is expressed predominantly on activated CD4+ T cells [1-3]. The interaction between CD40 and CD154 is critical for both the humoral and cellular immune responses. Humans with mutations in CD154 develop a severe form of immunodeficiency known as hyper IgM syndrome, characterised by elevated serum IgM, a failure to class switch in response to T cell-dependent antigens and an enhanced susceptibility to infection by opportunistic pathogens [1-3]. In vitro, signalling via CD40 drives the proliferation of B cells, protects against antigen receptor-induced apoptosis, and in the presence of cytokines, induces antibody class switching [1-3]. In addition, CD40 stimulation upregulates the expression of surface molecules such as MHC class I, MHC class II, ICAM-1, CD80 and CD86 [4-6] and enhances the production of cytokines such as IL-6, IL-10, IL-12 and TNF-α [1]. The ability of agonistic CD40 antibodies to trigger an effective anti-tumour immune response [7], highlights the importance of this molecule during antigen priming of cytotoxic T lymphocytes in vivo [8].

Engagement of CD40 by CD154 or anti-CD40 antibody, results in the recruitment of TNF receptor associated factors (TRAF) and activation of the constitutively associated tyrosine kinase, Jak3, the outcome of which is activation of the transcription factors NF-κB, c-Jun, NF-AT and STAT3/6 [3]. Like other members of the TNF superfamily, the extracellular domain of CD154 forms a homotrimer [9] and soluble trimeric CD154 has been shown to be biologically active [10]. Furthermore, soluble trimeric CD154 is known to be released from activated T cells by proteolytic cleavage, but the physiological role of this form of CD154 in vivo remains unclear [11]. Trimerisation of some members of the TNFR superfamily including Fas, TNFR II and TRAIL receptors is not sufficient to trigger a response, and higher order oligomers, as would be expected to occur in the plasma membrane, may be required to achieve a more effective response [12-14]. In contrast, trimerisation of two other members of this superfamily, TNFR I and TWEAK receptor, appears to be sufficient to induce the full signaling response [13, 14]. A recent study has shown that cross-linking of soluble trimeric CD154 by an antibody enhanced its ability to induce proliferation of peripheral blood B cells [15], although the mechanism by which this enhancement is achieved was not determined. An enhancement in the activity of soluble trimeric CD154 upon antibody cross-linking could be due to either an increase in the avidity of CD154, thus prolonging the interaction with CD40, or an increase in the level of receptor clustering such that it allows downstream adaptor proteins to signal effectively via a proximity-induced mechanism. US patent (U.S. Pat. No. 5,716,805) in the name of Immunex Corporation, also describes the preparation of soluble proteins that can display a heterologous protein as a trimer.

### SUMMARY OF THE INVENTION

The inventors have appreciated that the effective biological activity of a protein can be enhanced if the protein number is increased at the site of action. In contrast to simply providing an increased amount of protein, the inventors have devised a protein framework that allows the multimerisation of active proteins, polypeptides or peptides on a single structure. Thus, the concentration, or clustering effect, of the protein, polypeptide or peptide at the desired site of action is significantly increased. The inventors have found that the biological activity of these active polypeptides is increased when presented in multimers greater than a trimer.

Accordingly, in a first aspect of the present invention there is provided a purified protein complex capable of displaying a plurality of active polypeptides, said complex having a framework domain comprising multiple linked subunits, each subunit being a multimer of two or more polypeptide chains, each polypeptide chain having an active polypeptide associated at their C-terminus.

It is preferable that the soluble protein complex has at least two subunits, preferably three subunits and even more preferably four subunits. The subunits are linked together via the N-termini of the polypeptide chains. It is also preferred that each subunit comprises at least a dimer, preferably a trimer or at least a trimer, of the polypeptide chain and heterologous active polypeptide. The inventors show herein that the protein complex of the invention provides a higher level of active polypeptide clustering than trimeric molecules tried in the prior art, e.g. U.S. Pat. No. 5,716,805. As a result of the high level of clustering, the biological activity of the active polypeptide is significantly increased.

The inventors have found that collectins may be used as a framework to display multiple proteins on a single structure. Collectins are ideal as these proteins contain multiple trimeric heads (c-type lectins). Examples of possible collectins include the following: SP-D, SP-A, mannan binding protein (MBP) and conglutinin. The collectins are a family of soluble mammalian proteins known to bind carbohydrate structures via their c-type lectin domains. It is preferable to derive the framework domain according to the present invention from collecting as their lectin domains can easily be replaced with the protein of interest (active polypeptide). In an embodiment of the present invention, the lectin domains of the collectins are replaced by members of the TNF ligand superfamily. This can be efficiently achieved because the TNF ligand superfamily (e.g. CD154) have a so called type II orientation, that is the same orientation of the c-type lectin domains on the collectins. This means that the new fusion proteins will have the correct orientation to bind to their receptors (e.g. members of the TNF receptors superfamily).

In a preferred embodiment of the present invention the collectin used to provide the framework domain is Lung surfactant protein-D (SPED). The SP-D polypeptide chain consists of an N-terminal region, which forms inter-chain disulphide bonds that stabilises the overall structure, a collagenous region, an α helical coiled-coil and a C-terminal lectin domain [20, 21]. The trimerisation of the lectin domains is mediated by the a helical coiled-coil, referred to as the neck region [21]. The present inventors have discovered that the structure of collectins, e.g. SP-D, can be used as a framework domain to display a plurality of active polypeptides on a single structure. By removing the C-terminal lectin domain, the active polypeptide of interest can be associated, e.g. as a fusion protein, with the polypeptide chain of the collectin. The a helical coiled-coil of each of the polypeptide chains initiates the mulimerisation of the polypeptide chains. In the case of SP-D, trimerisation occurs resulting in a subunit comprising three polypeptide chains each associated with the active polypeptide. Thus, three active polypeptides are located closely together as a multimer.

As the N-terminal domain of each polypeptide chain is capable of associating via disulphide bridges, the trimeric subunits are also complexed thereby increasing yet again the number of active polypeptides in a single structure. In the case of SP-D, the resulting complex comprises 12 polypeptide chains that associate together to form 4 trimeric subunits. Thus, the framework derived from SP-D is capable of displaying a multimer of 12 active polypeptides (a dodecamer) on a single homogenous soluble protein complex.

The active polypeptides may be any protein, polypeptide or peptide whose effectiveness may be enhanced by increasing their clustering at an active site by multimerisation. The active polypeptide will be heterologous to the protein, e.g. collectin, providing the framework domain. For example, the present inventors have shown that the effectiveness of CD154 in proliferating B cells is significantly increased when CD154 is presented as a dodecamer in accordance with the present invention as opposed to the trimeric ligand (see detailed description). Examples of other active polypeptides include ligands or receptors e.g. any member of the TNF superfamily or receptor superfamily (e.g. CD40, CD134L, CD134, CD153, CD30, FasL, Fas) (see Smith et al Cell 1994. 76, 959; and Ashkenazi et al Science 1998, 281: 1305-1308), or any protein, polypeptide or peptide having the same basic design as a TNF family member; antigens, including tumour antigens; and antibody fragments including antibody binding domains. Example antibody fragments, capable of binding an antigen or other binding partner are the Fab fragment consisting of the VL, VH, C1 and CH1 domains; the Fd fragment consisting of the VH and CH1 domains; the Fv fragment consisting of the VL and VH domains of a single arm of an antibody; the dAb fragment which consists of a VH domain; isolated CDR regions and F(ab′)₂ fragments, a bivalent fragment including two Fab fragments linked by a disulphide bridge at the hinge region. Single chain Fv fragments are also included.

In a second aspect of the present invention, there is provided a nucleic acid construct comprising nucleic acid sequence encoding a polypeptide chain derived from a collectin having an N-terminal linking domain, an a helical coiled-coil and a C-terminal heterologous active polypeptide. The construct is preferably nucleic acid sequence encoding a polypeptide chain of a collectin e.g. SP-D (see FIG. 7) where the sequence encoding the lectin binding domain has been removed and replaced by sequence encoding the active polypeptide (protein of interest). The invention also provides a nucleic acid expression vector comprising the nucleic acid construct described above. The invention further provides an expression vector comprising nucleic acid sequence encoding a polypeptide chain capable of multimerisation e.g. trimerisation, said polypeptide chain having an N-terminal linking domain, an a helical coiled-coil capable of multimerisation, and an insertion site where nucleic acid sequence encoding an active polypeptide may be inserted in the correct orientation so as to express a fusion protein comprising the N-terminal linking domain, the a helical coiled-coil and the active polypeptide.

The insertion site may comprises restriction enzyme site whereby sequence encoding the active polypeptide may be inserted using standard molecular techniques. In order to obtain expression of the nucleic acid sequences according to the second aspect of the present invention, the sequences can be incorporated in a vector having control sequences operably linked to the nucleic acid sequence to control its expression. The vectors may include other sequences such as promoters or enhancers to drive the expression of the nucleic acid construct including the inserted nucleic acid, or nucleic acid encoding secretion signals so that the polypeptide produced in the host cell is secreted from the cell. The encoded polypeptide chain including the active polypeptide can then be obtained by transforming the vectors into host cells in which the vector is functional, culturing the host cells so that the polypeptide is produced and recovering the polypeptide from the host cells or the surrounding medium.

Preferably, the expressed polypeptides will be allowed to multimerise within the cell prior to recovery. Prokaryotic and eukaryotic cells are used for this purpose in the art, including strains of E. coli, yeast, and eukaryotic cells such as COS or CHO cells so as to allow glycosylation.

Generally, nucleic acid according to the present invention is provided as an isolate, in isolated and/or purified form, or free or substantially free of material with which it is naturally associated, such as free or substantially free of nucleic acid flanking the gene in the human genome, except possibly one or more regulatory sequence(s) for expression. Nucleic acid may be wholly or partially synthetic and may include genomic DNA, cDNA or RNA. Where nucleic acid according to the invention includes RNA, reference to the sequence shown should be construed as reference to the RNA equivalent, with U substituted for T. The nucleic acid sequence of the invention may be derived from the sequence encoding a collectin subunit, e.g. SP-D (see FIG. 7) or the sequence may have been modified, e.g. by mutagenesis, so as to improve it's multimerisation ability. Thus, the nucleic acid sequence of the invention may differ from the known sequence for collecting, e.g. as shown in FIG. 7 by a change which is one or more of addition, insertion, deletion and substitution of one or more nucleotides of the sequence shown. Changes to a nucleotide sequence may result in an amino acid change at the protein level, or not, as determined by the genetic code. Thus, the invention includes nucleic acid sequence which is a mutant, variant, derivative or allele of the known collectin sequences or a mutant, variant, derivative or allele of the known collectin polypeptide sequence, see for example the sequence given in FIG. 7 for SP-D.

In a third aspect of the present invention, there is provided a method of producing a protein complex according to the first aspect of the invention. The method preferably includes expressing nucleic acid encoding the polypeptide chain including the active polypeptide (generally nucleic acid according to the invention). This may conveniently be achieved by growing a host cell in culture, containing such a vector, under appropriate natural physiological conditions which cause or allow expression of the polypeptide and allow the polypeptide chains to multimerise. Polypeptides may also be expressed in in vitro systems, such as reticulocyte lysate.

Systems for cloning and expression of a polypeptide in a variety of different host cells are well known. Suitable host cells include bacteria, eukaryotic cells such as mammalian and yeast, and baculovirus systems. Mammalian cell lines available in the art for expression of a heterologous polypeptide include Chinese hamster ovary cells, HeLa cells, baby hamster kidney cells, COS cells and many others. A common, preferred bacterial host is E. coli.

Suitable vectors can be chosen or constructed, containing appropriate regulatory sequences, including promoter sequences, terminator fragments, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992.

Thus, the present invention further provides a host cell containing nucleic acid as disclosed herein. The nucleic acid of the invention may be integrated into the genome (e.g. chromosome) of the host cell. Integration may be promoted by inclusion of sequences which promote recombination with the genome, in accordance with standard techniques. The nucleic acid may be on an extra-chromosomal vector within the cell.

Following production by expression, the multimerised polypeptide chain and associated (by fusion) active polypeptide complex, may be isolated and/or purified from the host cell and/or culture medium, as the case may be, and subsequently used as desired, e.g. in the formulation of a composition which may include one or more additional components, such as a pharmaceutical composition which includes one or more pharmaceutically acceptable excipients, vehicles or carriers.

In a fourth aspect of the present invention there is provided a method treating an individual (human or animal, preferably mammal) suffering from or at risk of suffering from a disease state, said method comprising the step of administering to said individual, a protein complex as described above.

The active polypeptide associated with the framework region of the protein complex will depend on the disease state to be treated. For example, the inventors describe herein the increased proliferation of B-cells following treatment with a protein complex comprising CD154. The inventors have found that there is a significant increase in the activation of B-cells, and increase in the proliferation of B cells and, importantly, an increase in the levels of expression of co-stimulatory molecules such as ICAM-1, CD86 and MHC II. Thus, the inventors have provided a method of enhancing the activation of the cellular and humoral immune system, said method comprising the steps of administering to an individual a therapeutically acceptable amount of a protein complex defined above, wherein the active polypeptide is a member of the TNF superfamily, e.g. CD154. However, methods of inducing cell death, including tumour cell death may be achieved by administering a protein complex according to the invention wherein the active polypeptide is FasL or TRAIL. These peptides are known to induce apoptosis and their effectiveness may be increased if presented in a multimerised form to the active site, e.g. in a form according to the invention. Other examples of active polypeptides include CD154 which can trigger cell death in epithelial carcinoma; antibody fragments that are capable of targeting a tumour antigen/receptor which can then trigger cell signalling, e.g. CD20; or antibody fragments which block a signalling pathway such as via epidermal growth factor receptor. Alternatively, the multimerised protein complex may be used to provide an adjuvant effect for a vaccine, so called “smart vaccines/adjuvant”.

An alternative use of the protein complex of the invention is a method of in vitro activation of immune cells, such as dendritic cells, in the presence of an antigen, e.g. a tumour antigen. Other cells that may be activated include APCs, B cells, monocytes, follicular dendritic cells, thymic epithelial cells, endothelial cells and epithelial cells. The activated immune cells may then be administered in the form of a medicament to a patient requiring stimulation of an immune response against said antigen. The tumour antigen may be conveniently provided as part of the tumour cells. In other words, by activating the immune cells, e.g. dendritic cells, in the presence of a tumour cell, knowledge of the actual tumour antigen is not required.

The protein complex of the invention can be formulated in pharmaceutical compositions. These compositions may comprise, in addition to one of the above substances, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

The protein complex according to the present invention is preferably given to an individual in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

Alternatively, targeting therapies may be used to deliver the active agent more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

A composition may be administered alone or in combination with other treatments, either simultaneously or sequentially dependent upon the condition to be treated.

Thus, the present invention further provides a pharmaceutical composition, including a vaccine, comprising a protein complex according to the invention and a pharmaceutically acceptable carrier and/or adjuvant. The invention can advantageously be used to aid in the vaccination of an individual against a pathogen, by boosting the immune response as shown herein with the TNF superfamily. Alternatively, the protein complex may be used to present an antigen in a clustered format. For example, the effectiveness of a vaccine often depends on how the antigen in presented to the individual's immune system. If the antigen could be presented in a more concentrated or clustered form, i.e. as the active polypeptide in accordance with the invention, then the vaccination process may be achieved successfully using less actual antigen.

The present invention also provides the use of a polypeptide chain including the active polypeptide, or the protein complex in the preparation of a medicament for treating a disease state, such as cancer.

Alternatively, the medicament may be vaccine which can be used to vaccinate an individual against a pathogen.

In a further aspect of the present invention, there is provided a kit for producing a protein complex as described above. The kit preferably comprises a container containing an expression cassette as shown in FIG. 7 and instructions as to how to insert a protein of interest into said cassette. The expression cassette may be part of a expression vector or plasmid.

The inventors have shown that the multimerised complex of the invention allows greater activity or effect of the active polypeptide/protein of interest than alternative trimers. They have particularly shown this to be the case in vitro. Thus, the kit which has the SP-D platform in an expression vector where any protein of interest (in nucleic acid, e.g. DNA form) which is compatible (e.g. in orientation) with members of the TNF family can be cloned into it. The oligomeric SP-D fusion protein can then be used for example for research purposes e.g. in activating cells, signalling studies, induction of cell death, and other general molecular and cellular studies. The oligomeric SP-D-fusion protein molecule may be used to activate cells in vitro before delivery of these cells to a patient e.g. activation of dendritic cells in the presence of a tumour cells in order to activate the patients own immune system against cancer.

The invention further provides the SP-D platform (see FIG. 7) with certain modifications aimed at improving its properties. For example, the SP-D polypeptide chain (including glycosylation) may be engineered for the purpose of improving the half life of the protein in vivo and interaction with any receptors. Tags may also be introduced into the SP-D platform, such as FLAG, Poly His, c-myc, V5 epitope, or any other epitope tag for the purpose of purification and detection. Such tags, epitope tags and their corresponding antibodies may be provided in the kit of the present invention.

Aspects and embodiments of the present invention will now be illustrated, by way of example, with reference to the accompanying figures. Further aspects and embodiments will be apparent to those skilled in the art. All documents mentioned in this text are incorporated herein by reference.

### BRIEF DESCRIPTION OF THE DRAWINGS

FIG. 1. (A) Schematic representation of tCD154 and SP-D-CD154. (B) SDS-PAGE analysis on a 10% gel of purified SP-D-CD154 (lanes 1 and 3) and tCD154 (lanes 2 and 4) under non-reducing (lanes 1 and 2) and reducing conditions (lanes 3 and 4). Proteins (6 μg/lane) were visualised by Coommassie blue staining.

FIG. 2. Size exclusion chromatography of purified SPD-CD154 (A) and tCD154 (B). Proteins (16-70 μg) were analysed on Zorbax GF-250 HPLC column at a flow rate of 0.4 ml/min using phosphate buffer (0.2 M, pH 7.0, containing 1 M dimethyl formamide). Proteins were detected at 280 nm.

FIG. 3. Proliferation of splenic B cells following activation with SP-D-C154 (●) or tCD154 (∘). Proliferation was determined by measurement of [³H] thymidine incorporation after 88 h of culture. Error bars indicate SEM of triplicate wells.

FIG. 4. Flow cytomeric analysis of splenic cells following 24 h of activation with SP-D-C154 or tCD154. (A) The expression of ICAM-1, CD86 and MHC II is shown on untreated (i), SP-D-CD154 (ii) or tCD154 (iii) treated cells. The cells were also stained with the B cell marker CD19. (B) Forward scatter analysis of CD19+ untreated cells (thin solid line), SP-D-CD154 (thick solid line) or tCD154 (dotted line) treated cells.

FIG. 5. Detection of phosphorylated (A) and total (B) IκBα in cell lysates by Western blotting. Splenic cells were activated (10-60 min) and lysates were prepared as described in the Materials and methods.

FIG. 6. Real time analysis of the binding of (A) tCD154 (250 nM) and (B) SP-D-CD154 (125 nM) to CD40-Fc fusion protein using the BIAcore™ biosensor. Various concentrations of tCD154 or SP-D-CD154 (31.3-250 nM) were injected before and after injection of CD40-Fc at a flow rate of 5 μl/min.

FIG. 7. The dodacameric SPD expression cassette. The protein of interest (active polypeptide) can be cloned in frame using the EcoR I and/or the BanHI sites. The cassette is then subcloned into a mammalian expression vector, such as pEE14.

### DETAILED DESCRIPTION OF THE INVENTION

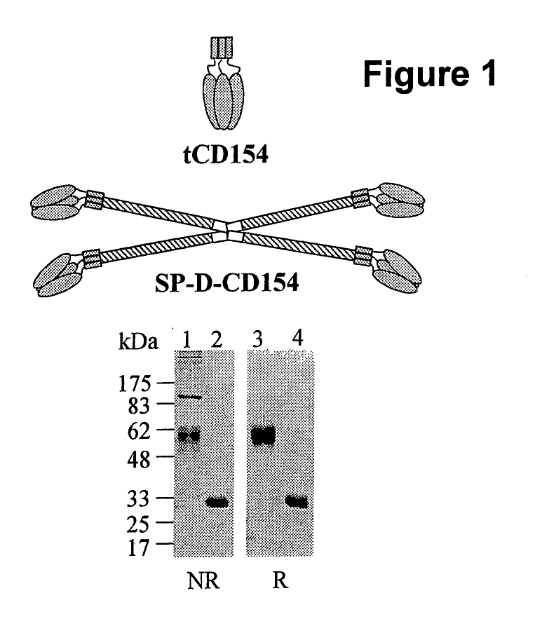

Specifically, and by way of example, the present inventors have generated two forms of soluble CD154 (FIG. 1A); the first is a novel dodecameric fusion protein between lung surfactant protein-D (SP-D) and CD154 (SP-D-CD154), and the second is a trimeric form of CD154 (tCD154). These two forms of CD154 allowed the direct investigation of the effect of CD40 oligomerisation on the downstream signalling events without the use of cross-linking antibodies. Moreover, to gain insights into the mechanism by which multimerisation enhances the biological activity of CD154, the affinity and kinetics of the interaction between soluble trimeric and dodecameric forms of CD154 and CD40 were determined.

Results and Discussion

Expression of Soluble Trimeric and Dodcameric CD154

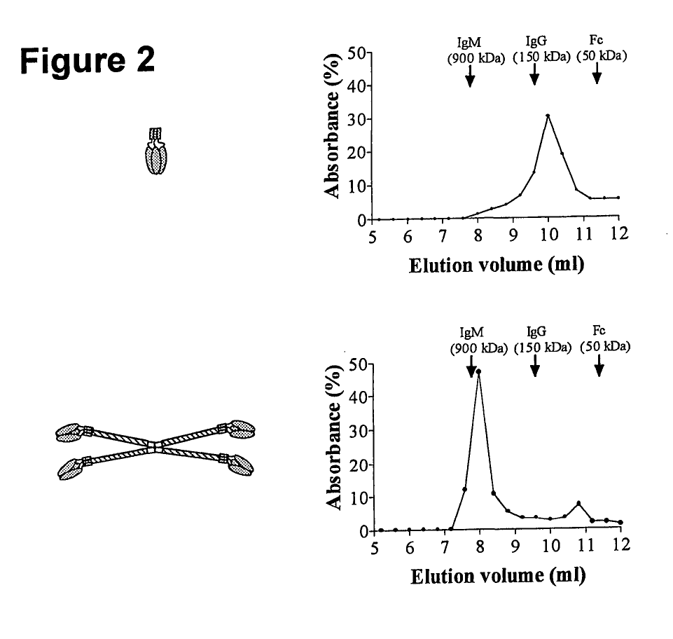

To express a dodecameric form of CD154, the lectin domains of SP-D were replaced by the C-terminal extracellular domain of murine CD154. SP-D is a C-type lectin produced by epithelial cells, mainly in the lung, that preferentially forms dodecamers, consisting of four trimeric subunits (FIG. 1A) [16]. SP-D binds to pathogenic micro-organisms in the lung and enhances their uptake and killing by alveolar macrophages and neutrophils [17]. The lectin domains of SP-D perform a dual function; the binding to carbohydrate structures on invading micro-organisms as well as the interaction with receptors on cells of the innate immune system [17-19]. The SP-D polypeptide chain consists of an N-terminal region, which forms inter-chain disulphide bonds that stabilises the overall structure, a collagenous region, an a helical coiled-coil and a C-terminal lectin domain [20, 21]. The trimerisation of the lectin domains is mediated by the a helical coiled-coil, referred to as the neck region [21]. The SP-D-CD154 fusion protein preserves the orientation of CD154 with respect to CD40 binding and thus mimics the orientation (type II) of membrane-bound CD154. A construct expressing soluble trimeric CD154 (tCD154) was also prepared which consisted of the extracellular domain of CD154 fused at its N-terminus to the neck region of SP-D. Analysis of the purified SP-D-CD154 and tCD154 by SDS-PAGE under reducing conditions revealed bands corresponding to proteins with a molecular mass of ˜58 and ˜30 kDa, respectively (FIG. 1B). These values are consistent with the predicted molecular mass of the polypeptide chains (tCD154, 28 kDa; SP-D-CD154, 48 kDa), plus 1 (tCD154) or 2 (SP-D-CD154) typical N-linked carbohydrates. Under non-reducing conditions, SP-D-CD154 gave four other bands in addition to the ˜58 kDa band, corresponding to higher molecular mass proteins (>58 kDa), consistent with the presence of inter-chain disulphide bonds (FIG. 1B). A previous study has shown that the substitution of the two conserved cysteine residues within the N-terminal region of SP-D with serine resulted in the production of exclusively trimeric form of SP-D, suggesting that these residues are required for the assembly of the four trimeric subunits into a dodecamer [20]. The apparent molecular mass of SP-D-CD154 determined by size-exclusion chromatography under non-denaturing conditions was ˜600 kDa, consistent with assembly of SP-D-CD154 into a dodecamer (FIG. 2A). Under the same chromatography conditions tCD154 had an apparent molecular mass of ˜100 kDa, suggesting that it forms a non-covalent homotrimer (FIG. 2B).

Oligomeric Requirement of CD154 for the Induction of B Cell Proliferation and Expression of ICAM-1, CD86 and MHC Class II

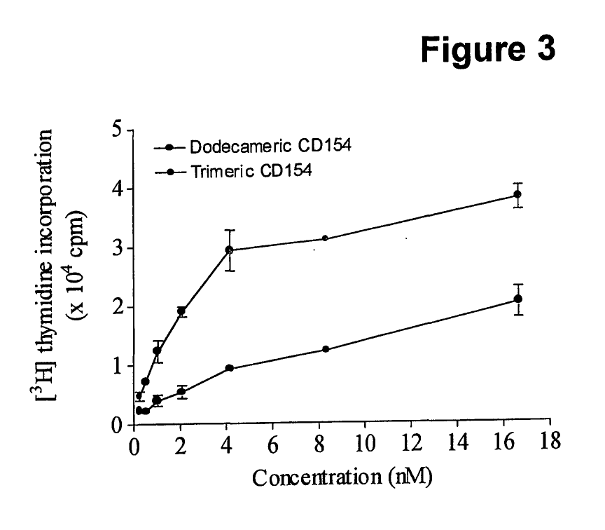

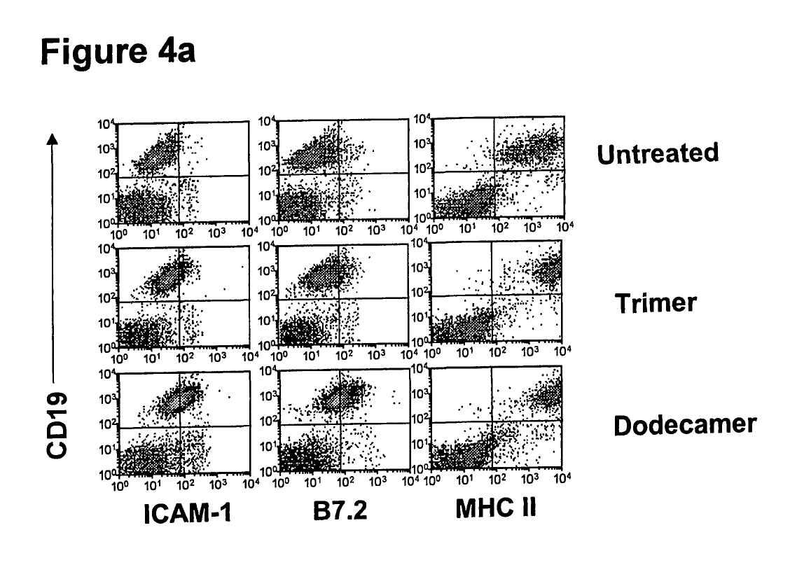

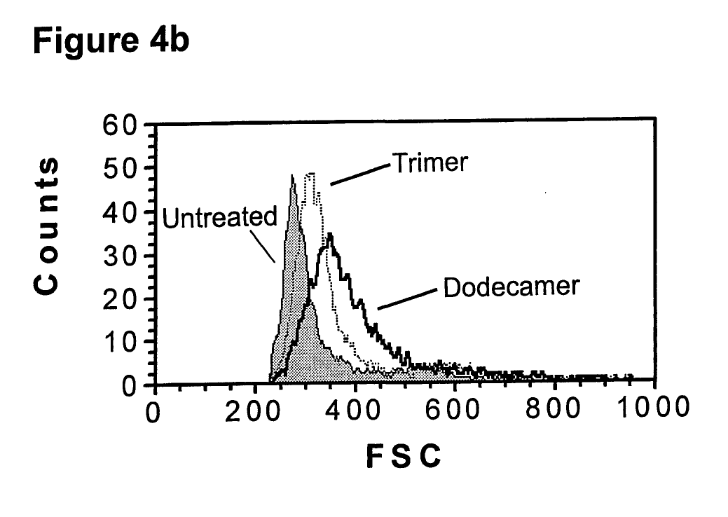

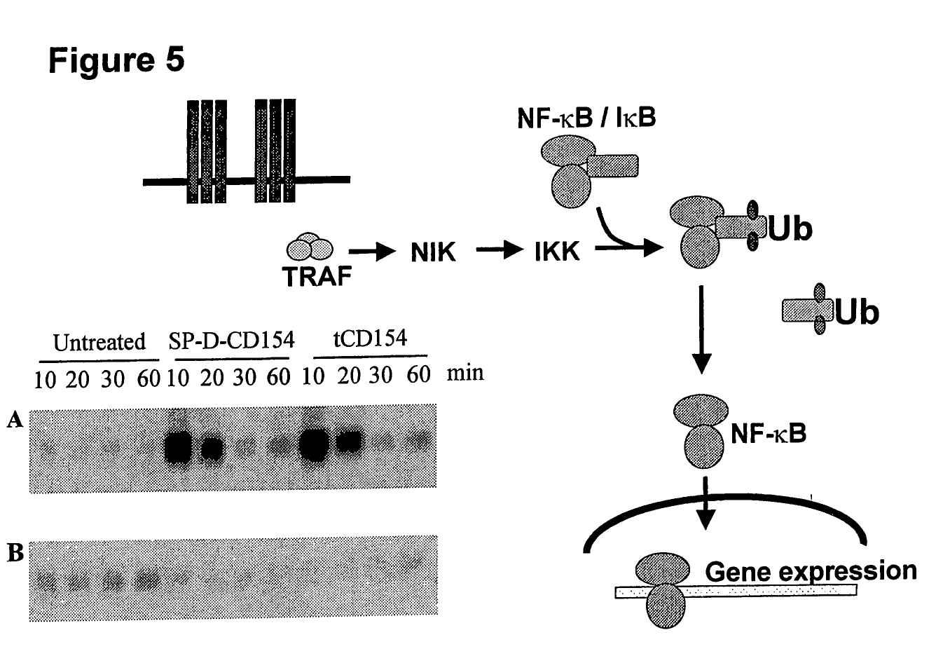

Both tCD154 and SP-D-CD154 induced the proliferation of murine splenic B cells in a concentration dependent manner (FIG. 3). This effect was observed with either whole splenic cultures, or purified B cells (data not shown). Multimeric SP-D-CD154 was ˜8-fold more potent than tCD154 in inducing B cell proliferation (FIG. 3). Furthermore, the proliferative response elicited by SP-D-CD154 or tCD154 was completely abolished by the addition of anti-CD154 mAb (MR1), confirming that this response is entirely dependent on CD154 and not any other part of the fusion protein (data not shown). The inventors then examined it other CD40-mediated functions are also influenced by the oligomeric nature of CD154. CD40 signaling upregulates the expression of costimulatory molecules on B cells and other antigen presenting cells, a process required for the priming and activation of both CD4 and CD8 T cells [4-6, 8]. The inventors analysed the expression of ICAM-1, CD86 and MHC class II on B cells 24 hours after incubation with either tCD154 or SP-D-CD154 (5 nM). Both tCD154 and SP-D-CD154 triggered upregulation of ICAM-1, CD86 and MHC class II, however when compared to tCD154, SP-D-CD154 consistently induced higher levels of ICAM-1 and CD86 expression (FIG. 4A). When compared to untreated cells, a 3.8- and 3.6-fold increase in the level of ICAM-1 and CD86, respectively, were obtained using SP-D-CD154, whereas stimulation with tCD154 produced a 2- and 1.4-fold increase in the level of ICAM-1 and CD86, respectively. In contrast, tCD154 and SP-D-CD154 induced similar levels of MHC class II expression (FIG. 4A). Analysis of the forward scatter of B cells (FIG. 4B), a measure of their relative size and activation status, revealed that activation with tCD154 triggered only a small increase in their size (mean forward scatter=368), when compared to cells activated with SP-D-CD154 (mean forward scatter=409). The inventors addressed whether the differences in the activities of SP-D-CD154 and tCD154 can be attributed to the activation of NF-κB. Oligomerization of CD40 results in the recruitment of several members of the TRAF family leading to the activation of NF-κB [1]. NF-κB is normally sequestered in the cytoplasm through interaction with IκB proteins [22]. Phosporylation of IκB proteins leads to their degradation via a proteosome-mediated pathway, resulting in the release and translocation of NF-κB into the nucleus, where it can activate the transcription of target genes [22]. The inventors' results demonstrate that both tCD154 and SP-D-CD154 were equally effective in inducing rapid phosphorylation of IκBα (followed by its degradation (FIG. 5). These results suggest that the differences in the downstream activities of SP-D-CD154 and tCD154 are unlikely to be due to differential IκBα phosphorylation, and imply the involvement of other CD40 signalling pathways, such as the activation of Jak3/STAT [23], c-Jun N-terminal kinase [24], or p38 mitogen activated protein kinase (MAPK) [25]. The observation that tCD154 and SP-D-CD154 were equally effective in upregulating the expression of MHC class II in B cells (FIG. 4A) indicates that the signalling pathways that mediate this process are likely to be distinct from those required for triggering the proliferation, or the expression of ICAM-1 and CD86. In agreement with this, an inhibitor of p38 MAPK was shown to inhibit CD40-induced B cell proliferation and upregulation of ICAM-1 expression, but not CD40, CD95, DR3, TRAF1/4 or cIAP2 expression, suggesting that CD40-induced functions are mediated by different signaling pathways [25].

tCD154 Binds to CD40 with High Apparent Affinity

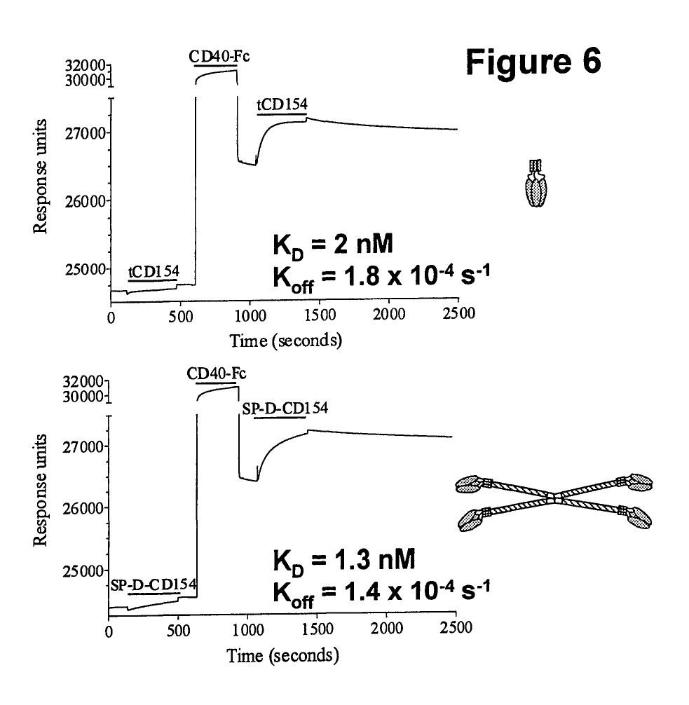

The lower activity of tCD154 when compared with SP-D-CD154 could be the result of its relatively low affinity for CD40. To address this question the inventors analysed the affinity and kinetics of the interaction between tCD154 or SP-D-CD154 and CD40 using the BIAcore™ biosensor, which measures protein-protein interaction in real time. A murine anti-human Fc mAb was covalently coupled to the dextran matrix, and either tCD154 or SPD-CD154 was then injected over this mAb in order to determine the level of non-specific binding. Specific binding was determined by first injecting murine CD40-human Fc fusion protein which bound to the immobilised anti-human Fc mAb, and then injecting (31.3-250 nM) tCD154 or SPD-CD154 (FIG. 6). Co-injection of the anti-CD154 mAb MR1 and tCD154 or SP-D-CD154 completely abolished the binding of tCD154 or SP-D-CD154 to CD40 (data not shown). The association (kₐ) and dissociation (k_{d}) rate constants were determined using the BIAevaluation 2.1 software (BIACORE). tCD154 bound to CD40 with high apparent affinity (K_{D}=2 nM) and dissociated very slowly (k_{d}=1.8×10⁻⁴s⁻¹; t_{1/2} ˜64 min). Using the same technique, the inventors have previously shown [26] that soluble trimeric OX40 ligand, a member of the TNF superfamily, binds to its receptor with a similar apparent affinity (K_{D}=3.8 nM). SP-D-CD154 also bound to CD40 with a high apparent affinity (K_{D}=1.3 nM), and dissociated with similar kinetics (k_{d}=1.4×10⁻⁴ s⁻¹; t_{1/2}˜83 min) to tCD154. These results suggest that the higher activity of SP-D-CD154 when compared to tCD154 is unlikely to be due to differences in their apparent affinity (avidity) for CD40, since both soluble forms of CD154 bound to CD40 with high avidity and dissociated very slowly following binding. SP-D-CD154 can potentially bind to twelve CD40 molecules, compared to three molecules with tCD154 [9], implying that the extent of receptor oligomerisation may influence the signals generated by CD40. Within the X-shaped SP-D molecule, the adjacent arms are separated by a distance of either ˜20 nm or ˜90 nm (FIG. 1A) [16]. Thus if all four arms of SP-D-CD154 engage cell surface-expressed CD40, two clusters are likely to form each with six closely associated CD40 molecules. The cytoplasmic adapter proteins, TRAF2 and TRAF3 have been shown to bind to a trimerised form of the cytoplasmic tail of CD40 with low affinity (K_{D}=3-13 μM), and the affinity for the interaction with TRAF6 was estimated to be even lower, although the K_{D} of this interaction was not determined [27]. Therefore, the close association of six CD40 receptors may provide a high avidity platform, which facilitates a more stable interaction with downstream adapter proteins, such as the TRAFs. Alternatively, the association of six or more CD40 receptors into clusters may trigger signaling more effectively by a proximity induced mechanism as described for the activation of caspase 8 [28].

Taken together, the data presented here demonstrates conclusively that tCD154, which binds to CD40 with high apparent affinity, is sufficient to trigger signalling in B cells, however, higher order oligomers provide a more potent stimulus. Recent studies have shown that during cell-cell interaction, receptors and ligands segregate within contact zones resulting in the formation of supramolecular clusters [29]. If similar clusters exist between CD154 and CD40 during cell-cell interaction, then this would generate high order oligomeric complexes that are more effective in signalling than single trimeric units. Finally, the strategy described here could be adapted for other proteins and particularly for other members of the TNF superfamily, where trimeric forms are known to be ineffective [12-14]. The use of the SP-D multimerisation platform for the construction of soluble and highly active members of the TNF superfamily may prove to be particularly useful for the generation of immunotherapeutic agents. One potential candidate is the CD154 molecule itself, which is essential for the priming of cytotoxic T cell responses such as those required for the generation of a protective anti-tumour response [7].

Materials and Methods

Construction and Expression of SP-D-CD154 and tCD154

The region encoding amino acid (aa) residues 1-257 of SP-D was amplified from a plasmid containing full-length human SP-D. The 5′oligonucleotide introduced a XbaI site, and the 3′oligonucleotide incorporated a linker (GGGNS), an EcoRI site and a downstream BamHI site. The digested PCR fragment was ligated into pEE14 (Lonza Biologics) at the XbaI and BclI sites to produce pEE14/SP-D. The extracellular domain of CD154, (aa residues 50-260), was amplified using cDNA from 48 h concanavalin A activated mouse splenocytes, introducing 5′ and 3′ EcoRI sites. The PCR product was cloned into the EcoRI site of pEE14/SP-D. The predicted amino acid sequence at the junction between SP-D and the N-terminus of CD154 is (SP-D)LFPNG/GGGNS/LDKVE(CD154). A tCD154 construct encoding the rat CD4 leader, the α helical coiled-coil domain of SP-D (aa 223-257) and the extracellular domain of CD154 was prepared by a three-step overlaping PCR strategy. The PCR fragment was digested with HindIII and XbaI and ligated into pEE14. The expression constructs were transfected into CHO-K1 as described previously [30]. Recombinant proteins were purified from tissue culture supernatant by affinity chromatography using MR-1 mAb column [31]. tCD154 was further purified by size-exclusion chromatography using a Superdex™ 200 HR10/30 column (Amersham Pharmacia Biotech AB). The extinction coefficients at 280 nm, E_{(0.1%, 1 cm)} for SP-D-CD154 (0.453) and tCD154 (0.678) were estimated from the amino acid sequence using the ProtParam tool (www.expasy.ch [32]).

Measurement of [³H] Thymidine Incorporation

Mouse splenocytes (5×10⁵/ml) were cultured in RPMI 1640, 10% (v/v) foetal calf serum and 25 μM 2-ME using U-shaped 96 well plates. After 72 h of culture, wells were pulsed with 0.5 μCi of [³H] thymidine for the final 16 h of culture.

Analysis of the Expression of Cell Surface Molecules by Flow Cytometry

Mouse splenocytes (1.25×10⁶/ml) in 2 ml cultures were treated with either SP-D-CD154 or tCD154 (5 nM) or left untreated for 24 hours. Cells were incubated with PE-labelled anti-CD19 mAb (Serotec) and FITC-labelled mabs (10 μg/ml) to ICAM-1 (YN1.4.7), CD86 (GL-1), and MHC class II (N22) in PBS, 0.2% (w/v) BSA, 1% (v/v) mouse serum.

Analysis of IκB-α Phosphorylation

Mouse splenocytes (5×10⁵/ml) were cultured in serum free media with SP-D-CD154, tCD154 (5 nM) or media alone. Cells were lysed, and the equivalent of 5×10⁴ cells were analysed by SDS-PAGE. The levels of total and phophorylated IκB-α were detected by Western blotting (PhosphoPlus® IκB-α (Ser-32) Antibody Kit, New England Biolabs).

BIAcore™ Analysis

All experiments were performed at the indicated flow rates in Hepes buffered saline (150 mM NaCl, 0.005% (v/v) surfactant P20 (BIACORE), 10 mM Hepes, pH 7.4). mAbs were covalently bound to the carboxylated dextran matrix using the amine coupling kit (BIACORE) as described previously [26]. For analysis of the expression of SP-D-CD154 and tCD154 by CHO-K1 clones, culture supernatant was injected at 4 μl/min (7.5 min) over MR-1 mAb. To assess binding of SP-D-CD154 or tCD154 to murine CD40, CD40-human Fc fusion protein was first injected over covalently bound anti-human Fc mAb (SB2H2) at a flow rate of 5 μl/min (6 min) followed by injection (6 min) of SP-D-CD154 or tCD154 (31.3 nM-250 nM).

### SUMMARY

Communication between cells of the immune system through cell-cell interactions is critical for the initiation and maintenance of an appropriate immune response. Cell-cell interactions are mediated by glycoproteins (also known as receptors and ligands) that are anchored to the cell surface of immune cells normally through a stretch of hydrophobic residues known as the transmembrane domain. A signal within an immune cell is initiated when the extracellular domain of a specific receptor is bound to a specific glycoprotein known as the ligand. It is the extracellular domain of the ligand alone that is responsible for binding to the receptor, and as a result of this, a signalling cascade is initiated which may activate for example a lymphocyte to react against an invading organism. Such signals may be artificially induced, for example in order to enhance an immune response during vaccination, or to stimulate an immune response against certain diseases such as cancer, by providing an exogenous form of the stimulatory ligands. This can be achieved by preparing a soluble recombinant form of the ligand containing the extracellular receptor-binding domain, or a protein, such as an antibody fragment, that is capable of binding to the receptor and inducing signalling. Many receptors, such as members of the tumour necrosis (TNF) receptor superfamily, require clustering to mediate their signals. This is normally attained through presentation of the natural membrane-bound ligand in a highly multimeric fashion. Multimerisation is acquired at two different levels. First, certain ligands adopt a native oligomeric fold, for example trimers. Second, these ligands when presented on the cell surface appear as an array of highly multimeric proteins. This invention describes methods-to generate soluble proteins (ligands) that artificially mimic the highly multimeric natural membrane-bound forms, with the aim of using these proteins therapeutically to modulate immune responses.

The following is an example of how this technology may be applied to the CD40 ligand (also known as CD154) molecule. The same strategy could be applied to other members of this family of molecules including, CD27 ligand, CD30 ligand, CD95 ligand (Fas), CD134 ligand, CD137 ligand and TRAIL, all of which have been shown to have important roles in immune regulation as well as the control of survival and death of normal and malignant cells.

Application of the Technology to the CD40 Ligand Molecule

CD40 is a member of the TNF receptor superfamily and is expressed on a number of cells including B cells, various antigen presenting cells (APCs) fibroblasts, epithelial cells and endothelial cells. CD40 binds to CD154, a member of the TNF family that is expressed mainly on activated CD4⁺ T helper cells. CD40-CD154 interaction plays an important role in the generation of humoral and cellular immune responses. Mice that have been rendered deficient for CD40 or CD154 are immuno-compromised with respect to antibody production and Ig-class switching, and are unable to mount an effective response to infectious pathogens such as Leishmania. Humans that have mutations in DC154 develop a severe form of immunodeficiency, known as hyper IgM syndrome, that is characterised by high levels of IgM and low levels of IgA, IgE and IgG, the absence of germinal centres and the inability to mount a thymus-dependent humoral response. It is now clear that CD40 plays a critical role in activating APCs and is important for generating cytotoxic T lymphocytes (CTLs). Recent date show that during an immune response an activation signal is delivered to the APC via CD40 through interaction with CD154 on the T helper cell. This activation signal “conditions” the APCs and empowers them to stimulate CTLs. Although the nature of the “conditioning” induced by CD40-triggering on APCs is not fully understood, it probably involves a combination of improved antigen processing, increased expression of co-stimulatory and adhesion molecules and up-regulation of cytokine production. There is now convincing evidence to show that professional APCs, such as dendritic cells are capable of presenting tumour antigens to CTLs by a process known as cross-priming.

An antibody that cross-links CD40 on the surface of APCs can therefore replace the requirement of the help provided by CD4⁺ T helper cells expressing CD154. These observations suggest that in cases where T helper responses are compromised or absent, as the case may be in cancer, stimulation of CD40 on professional APCs could be used to provoke an immune response. Recently, Nakajima et al. have shown that transfection of the otherwise non-immunogenic P815 mastocytoma with the cDNA for membrane-bound full length CD154 triggers a specific immune response that results in their prompt rejection. Futhermore these CD154 expressing P815 tumour cells were able to elicit protective immunity against subsequent challenge with parental P815 cells, thus leading the authors to suggest that this approach could be useful as a new strategy for immuno-gene therapy for tumours.

It has recently been demonstrated that treatment of B-cell lymphoma-bearing mice with CD40 monoclonal antibody (mAb) generates a rapid CD4⁺ T cell independent CTL response capable of eradicating the syngeneic tumour cells. The therapeutic activity of the CD40 mAb is dependent on the presence of an intact Fc region, which is required for the cross-linking of several CD40 molecules on the APCs. These results are consistent with the current understanding of the requirements for signalling by members of the TNF receptor superfamily. However, the immunogenicity of either murine or chimerized mAbs together with any potential immunotoxicity resulting from cross-linking of Fc receptors on effector cells will very likely preclude their use in man.

A method for the Preparation of Soluble Recombinant Highly Multimeric CD154

The extracellular domain of CD154 has been shown to form a homotrimer and to adopt a similar fold to that of TNF-α and lymphotoxin-α. A number of recent studies utilising soluble TNF-α, Fas ligand and CD30 ligand (Hargreaves and Al-Shamkhani, unpublished) have suggested that further cross-linking of the timers may be necessary to produce the full biological activity of the natural membrane-bound form. Therefore, a novel highly multimeric soluble fusion protein consisting of the extracellular domain of CD154 and specific domains of lung surfactant protein-D was produced in Chinese hamster ovary cells (CHO). This chimeric protein is likely to be non-immunogenic as all of its components will be of human origin. The SPD-CD154 chimeric protein was purified by affinity chromatography.

Characterisation by gel filtration chromatography and SDS-PAGE shows that the purified protein is a homogenous product consisting of 12 polypeptide chains that associate together to form 4 trimeric CD40 ligand subunits. An in vitro B cell proliferation assay was used to assess the biological activity of SPD-CD154. The inventors have shown that SPD-CD154 is extremely potent in inducing the proliferation of B cells. When compared with membrane-bound ligand, as little as 2 μg/ml of SPD-CD154 produced an equivalent level of proliferation to that obtained by as many as 50 000 cells expressing membrane-bound CD154. These results confirm that SPD-CD154 is biologically active and can replace the signal normally provided by the natural membrane-bound ligand.

### REFERENCES

1 Vogel, L. A. and Noelle, R. J., CD40 and its crucial role as a member of the TNFR family. Semin Immunol 1998. 10: 435-442.

2 Grewal, I. S. and Flavell, R. A., CD40 and CD154 in cell-mediated immunity. Annu Rev Immunol 1998. 16: 111-135.

3 van Kooten, C. and Banchereau, J., CD40-CD40 ligand. J Leukoc Biol 2000. 67: 2-17.

4 Kiener, P. A., Moran-Davis, P., Rankin, B. M., Wahl, A. F., Aruffo, A. and Hollenbaugh, D., Stimulation of CD40 with purified soluble gp39 induces proinflammatory responses in human monocytes. J Immunol 1995. 155: 4917-4925.

5 Wu, Y., Xu, J., Shinde, S., Grewal, I., Henderson, T., Flavell, R. A. and Liu, Y., Rapid induction of a novel costimulatory activity on B cells by CD40 ligand. Curr Biol 1995. 5: 1303-1311.

6 Shinde, S., Wu, Y., Guo, Y., Niu, Q., Xu, J., Grewal, I. S., Flavell, R. and Liu, Y., CD40L is important for induction of, but not response to, costimulatory activity. ICAM-1 as the second costimulatory molecule rapidly up-regulated by CD40L. J Immunol 1996. 157: 2764-2768.

7 French, R. R., Chan, H. T., Tutt, A. L. and Glennie, M. J., CD40 antibody evokes a cytotoxic T-cell response that eradicates lymphoma and bypasses T-cell help. Nat Med 1999. 5: 548-553.

8 Ridge, J. P., Di Rosa, F. and Matzinger, P., A conditioned dendritic cell can be a temporal bridge between a CD4+T-helper and a T-killer cell. Nature 1998. 393: 474-478.

9 Karpusas, M., Hsu, Y. M., Wang, J. H., Thompson, J., Lederman, S., Chess, L. and Thomas, D., 2 A crystal structure of an extracellular fragment of human CD40 ligand. Structure 1995. 3: 1031-1039.

10 Mazzei, G. J., Edgerton, M. D., Losberger, C., Lecoanet-Henchoz, S., Graber, P., Durandy, A., Gauchat, J. F., Bernard, A., Allet, B. and Bonnefoy, J. Y., Recombinant soluble trimeric CD40 ligand is biologically active. J Biol Chem 1995. 270: 7025-7028.

11 Pietravalle, F., Lecoanet-Henchoz, S., Aubry, J. P., Elson, G., Bonnefoy, J. Y. and Gauchat, J. F., Cleavage of membrane-bound CD40 ligand is not required for inducing B cell proliferation and differentiation. Eur J Immunol 1996. 26: 725-728.

12 Grell, M., Douni, E., Wajant, H., Lohden, M., Clauss, M., Maxeiner, B., Georgopoulos, S., Lesslauer, W., Kollias, G., Pfizenmaier, K. and et al., The transmembrane form of tumor necrosis factor is the prime activating ligand of the 80 kDa tumor necrosis factor receptor. Cell 1995. 83: 793-802.

13 Tanaka, M., Itai, T., Adachi, M. and Nagata, S., Downregulation of Fas ligand by shedding. Nat Med 1998. 4: 31-36.

14 Schneider, P., Holler, N., Bodmer, J. L., Hahne, M., Frei, K., Fontana, A. and Tschopp, J., Conversion of membrane-bound Fas (CD95) ligand to its soluble form is associated with downregulation of its proapoptotic activity and loss of liver toxicity. J Exp Med 1998. 187: 1205-1213.

15 Pound, J. D., Challa, A., Holder, M. J., Armitage, R. J., Dower, S. K., Fanslow, W. C., Kikutani, H., Paulie, S., Gregory, C. D. and Gordon, J., Minimal cross-linking and epitope requirements for CD40-dependent suppression of apoptosis contrast with those for promotion of the cell cycle and homotypic adhesions in human B cells. Int Immunol 1999. 11: 11-20.

16 Crouch, E., Persson, A., Chang, D. and Heuser, J., Molecular structure of pulmonary surfactant protein D (SP-D). J Biol Chem 1994. 269: 17311-17319.

17 Lawson, P. R. and Reid, K. B., The roles of surfactant proteins A and D in innate immunity. Immunol Rev 2000. 173: 66-78.

18 Holmskov, U., Lawson, P., Teisner, B., Tornoe, I., Willis, A. C., Morgan, C., Koch, C. and Reid, K. B., Isolation and characterization of a new member of the scavenger receptor superfamily, glycoprotein-340 (gp-340), as a lung surfactant protein-D binding molecule. J Biol Chem 1997. 272: 13743-13749.

19 Sano, H., Chiba, H., Iwaki, D., Sohma, H., Voelker, D. R. and Kuroki, Y., Surfactant proteins A and D bind CD14 by different mechanisms. J Biol Chem 2000. 275: 22442-22451.

20 Brown-Augsburger, P., Hartshorn, K., Chang, D., Rust, K., Fliszar, C., Welgus, H. G. and Crouch, E. C., Site-directed mutagenesis of Cys-15 and Cys-20 of pulmonary surfactant protein D. Expression of a trimeric protein with altered anti-viral properties. J Biol Chem 1996. 271: 13724-13730.

21 Hakansson, K., Lim, N. K., Hoppe, H. J. and Reid, K. B., Crystal structure of the trimeric alpha-helical coiled-coil and the three lectin domains of human lung surfactant protein D. Structure Fold Des 1999. 7: 255-264.

22 Karin, M. and Ben-Neriah, Y., Phosphorylation meets ubiquitination: the control of NF-[kappa]B activity. Annu Rev Immunol 2000. 18: 621-663.

23 Hanissian, S. H. and Geha, R. S., Jak3 is associated with CD40 and is critical for CD40 induction of gene expression in B cells. Immunity 1997. 6: 379-387.

24 Pullen, S. S., Dang, T. T., Crute, J. J. and Kehry, M. R., CD40 signaling through tumor necrosis factor receptor-associated factors (TRAFs). Binding site specificity and activation of downstream pathways by distinct TRAFs. J Biol Chem 1999. 274: 14246-14254.

25 Craxton, A., Shu, G., Graves, J. D., Saklatvala, J., Krebs, E. G. and Clark, E. A., p38 MAPK is required for CD40-induced gene expression and proliferation in B lymphocytes. J Immunol 1998. 161: 3225-3236.

26 Al-Shamkhani, A., Mallett, S., Brown, M. H., James, W. and Barclay, A. N., Affinity and kinetics of the interaction between soluble trimeric OX40 ligand, a member of the tumor necrosis factor superfamily, and its receptor OX40 on activated T cells. J Biol Chem 1997. 272: 5275-5282.

27 Pullen, S. S., Labadia, M. E., Ingraham, R. H., McWhirter, S. M., Everdeen, D. S., Alber, T., Crute, J. J. and Kehry, M. R., High-affinity interactions of tumor necrosis factor receptor-associated factors (TRAFs) and CD40 require TRAF trimerization and CD40 multimerization. Biochemistry 1999. 38: 10168-10177.

28 Muzio, M., Stockwell, B. R., Stennicke, H. R., Salvesen, G. S. and Dixit, V. M., An induced proximity model for caspase-8 activation. J Biol Chem 1998. 273: 2926-2930.

29 van der Merwe, P. A., Davis, S. J., Shaw, A. S. and Dustin, M. L., Cytoskeletal polarization and redistribution of cell-surface molecules during T cell antigen recognition. Sem Immunol 2000. 12: 521.

30 Davis, S. J., Ward, H. A., Puklavec, M. J., Willis, A. C., Williams, A. P. and Barclay, A. N., High level expression in Chinese hamster ovary cells of soluble forms of CD4 T lymphocyte glycoprotein including glycosylation variants. J Biol Chem 1990. 265: 10410-10418.

31 Noelle, R. J., Roy, M., Shepherd, D. M., Stamenkovic, I., Ledbetter, J. A. and Aruffo, A., A 39-kDa protein on activated helper T cells binds CD40 and transduces the signal for cognate activation of B cells. Proc Natl Acad Sci USA 1992. 89: 6550-6554.

32 Gill, S. C. and von Hippel, P. H., Calculation of protein extinction coefficients from amino acid sequence data. Anal Biochem 1989. 182: 319-326.

1. A protein complex capable of displaying a plurality of active polypeptides, said complex having a framework domain comprising at least two linked subunits, each subunit being a multimer of two or more polypeptide chains, each polypeptide chain having an active polypeptide associated at its C-terminus.

2. A protein complex according to claim 1 wherein the active polypeptide and the polypeptide chain is a fusion protein.

3. A protein complex according to claim 1 or claim 2 wherein each subunit comprises trimeric polypeptide chains.

4. A protein complex according to claim 3 wherein the framework domain comprises four subunits linked by the N-terminals of the trimeric polypeptide chains.

5. A protein complex according to any one of the preceding claims wherein the framework domain is derived from a collectin.

6. A protein complex according to claim 5 wherein the framework domain is derived from SP-D, SP-A, MBP or conglutinin.

7. A protein complex according to claim 6 wherein the framework domain is derived from SP-D.

8. A protein complex according to any one of the preceding claims wherein the active polypeptide is CD154, Cd40, CD134L, CD134, CD153, CD30, FasL, or Fas.

9. An isolated nucleic acid construct comprising nucleic acid sequence encoding a fusion protein comprising an N-terminal associating domain, an a helical coiled-coil, and a C-terminal active polypeptide.

10. An isolated nucleic acid construct according to claim 9 wherein the nucleic acid sequence encoding the N-terminal associating domain and the a helical coiled-coil is derived from nucleic acid encoding a collectin polypeptide chain.

11. An isolated nucleic acid construct according to claim 10 wherein the collectin is SP-D.

12. An expression vector comprising a nucleic acid construct according to any one of claims 9 to 11.

13. An expression vector comprising nucleic acid sequence encoding a collectin polypeptide chain capable of multimerisation wherein the sequence encoding the lectin binding domain is replaced with an insertion site thereby allowing insertion of additional nucleic acid sequence encoding an active polypeptide.

14. An expression vector according to claim 13 wherein the collectin is SP-D.

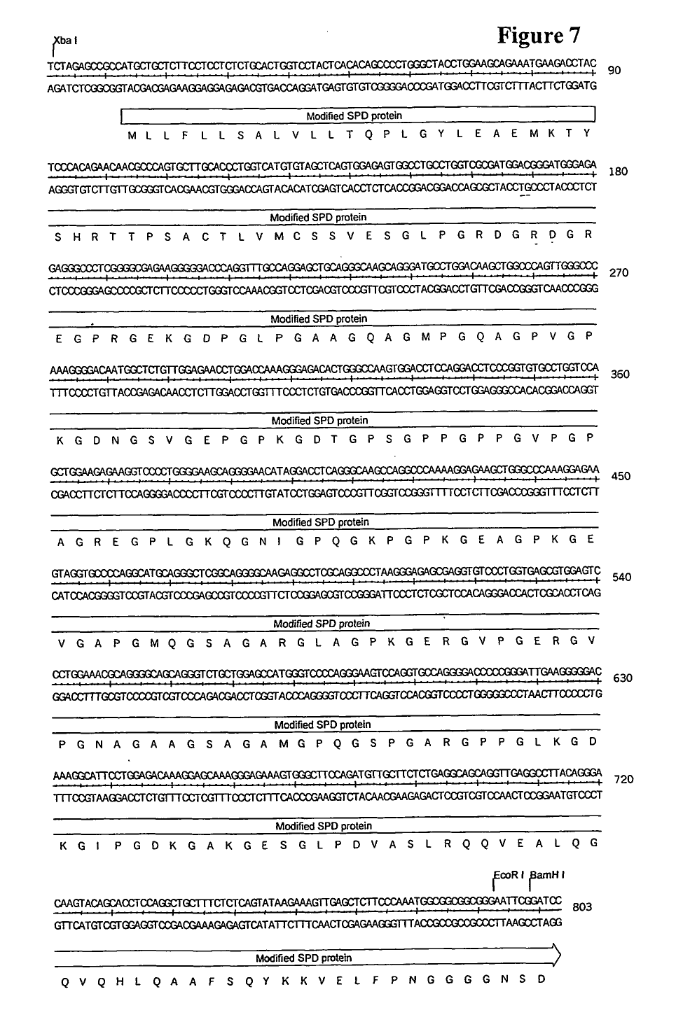

15. An expression vector according to claim 14 wherein the nucleic acid sequence is as shown in FIG. 7.

16. A host cell comprising a nucleic acid construct according to any one of claims 9 to 11 or an expression vector according to any one of claims 12 to 15.

17. A method of producing a protein complex according to any one of claims 1 to 8 comprising culturing the host cells of claim 16 so that polypeptide chains produced each displaying an active polypeptide, and allowing said polypeptide chains to multimerise to form subunits and allowing said subunits to link to form said complex.

18. A method according to claim 17 comprising the further step of recovering the protein complex.

19. A pharmaceutical composition comprising a protein complex according to any one of claims 1 to 8 and a pharmaceutical acceptable carrier.

20. A method of stimulating of an immune cell in vitro comprising the steps of contacting said immune cell with a protein complex according to any one of claims 1 to 8 in the presence of an antigen, wherein said active polypeptide is a member of the TNF superfamily.

21. A method according to claim 20 wherein the immune cell is a dendritic cell.

22. A method according to claim 20 or claim 21 wherein the antigen is a tumour antigen and the active polypeptide is CD154.

23. A method of enhancing the activation of an immune response to an antigen in an individual, comprising the steps administering to said individual a protein complex according to any one of claim 1 to 8 wherein the active polypeptide is a member of the TNF superfamily.

24. A method according to claim 23 wherein the active polypeptide is CD154.

25. A kit for producing a protein complex according to any one of claim 1 to 8 comprising a container containing an expression cassette encoding an SP-D subunit containing restriction endonuclease site for insertion of an active polypeptide and instructions as to how to produce said protein complex.

26. A kit according to claim 25 wherein the expression cassette has the sequence as shown in FIG. 7.
